(19) United States
(12) Patent Application Publication
(10) Pub. No.: US 2022/0211034 A1
(43) Pub. Date: Jul. 7, 2022

(54) VARIOUS USES OF THE NANOPARTICULATE COMPOUND OF TITANIUM DIOXIDE FUNCTIONALIZED

(71) Applicants: Gabriela LEÓN GUTIÉRREZ, (US); Inmolecule International Limited, London (GB)

(72) Inventors: Gabriela LEÓN GUTIÉRREZ, Ciudad de Mexico (MX); Sergio Manuel LEÓN GUTIÉRREZ, Ciudad de Mexico (MX)

(73) Assignees: Inmolecule International Limited, London (GB); Gabriela LEÓN GUTIÉRREZ, Ciudad de Mexico (MX)

(21) Appl. No.: 17/600,910

(22) PCT Filed: Apr. 2, 2020
(86) PCT No.: PCT/IB2020/053120

§ 371 Date: Oct. 1, 2021

(30) Foreign Application Priority Data
Apr. 4, 2019 (MX) MX/A/2019/003969

(51) Int. Cl. A01N 25/26; A01N 25/24; A01N 59/16; A61K 9/16; A01P 1/00; A61P 31/00

(52) U.S. Cl. CPC A01N 25/26; A01N 25/24; A61P 31/00; A61K 9/1611; A01P 1/00; A01N 59/16

(57) ABSTRACT

The present invention refers to various uses of the compound of titanium dioxide modified with organic functional groups, inorganic radicals and herbal and/or fruit extracts adsorbed on its surface and pores, wherein said compound is used as: water disinfectant or purifier agent; biopesticide and post-harvest; preservative in preparation of industrialized hygienic, cosmetic and food products; inductor or activator of tissue regeneration; in the pharmaceutical industry by administering it through different systemic pathways, being effective on treatment and prevention of infection processes caused by viruses, bacteria, fungi, spores, mycobacteria and parasites; microbial agent, mixed with water; antineoplastic for battling pathogenic microorganisms; microbial agent in the livestock, cattle and aquaculture industries.

### FIELD OF INVENTION

The present invention refers to techniques and principles used in nanotechnology for the manipulation of matter at the atomic and molecular level for various purposes such as industrial or pharmaceutical, and more particularly, it refers to various uses of the nanoparticulate compound of titanium dioxide functionalized with functional organic groups, inorganic radicals and fruit and/or herbal extracts adsorbed on its surface and pores as described in Patent MX 339086.

### BACKGROUND OF INVENTION

Throughout history the use of antimicrobials has been recurrent, being used even in the most ancient cultures. These substances capable of eliminating microorganisms have been perfectioned and industrialized until our modern days. Today, the use of some of said substances has been prohibited in developed countries.

A microbial substance is one capable of eliminating or inhibiting the growth of fungi, bacteria and parasites. It does not contemplate the sporulated vegetative forms nor viruses. Antibodies belong to this classification. Notwithstanding, it should be noted that not all microbials developed in past centuries have had a broad spectrum nor they have contemplated viruses, spores or mycobacteria.

The historical event that marked the age of antibiotics was the discovery of penicillin, which set off and intense search of other natural or synthetic compounds that could be used to treat other pathogenic microorganisms. That search has been more difficult with some microorganisms than with others. Bacterial cells differ from human cells in many fundamental aspects, such that they offer more opportunities of developing new drugs. Instead, fungi, parasites and viruses share with human cells many metabolic pathways and structures, offering the researchers less therapeutic targets and bringing more toxicity risks for patients.

This set off was generated due to, after a few years of use of penicillin, the confirmation that it generated microbial resistance, it is to say, that there were bacterial strains capable of being immune thereto. With this, treatments effectivity is loss, not only in humans, but in the cattle and even in the agriculture use.

The proper and on time treatment with antimicrobials is the difference between life and death, or else, with chronic incapacities. However, the inadequate and excessive use of these substances have created microorganisms super resistant thereto, and this is the reason why there are antibiotics of fifth generation. Still with the application of these new generations of antibiotics, today said super resistant microorganisms defend themselves or they are not inhibited nor eliminated using an antimicrobial or even mixtures thereof.

Hence the importance of a new antimicrobial, which not only inhibits nor eliminate fungi, bacteria and parasites, but also eliminates or inhibits the growth of mycobacteria, spores or the deactivation of viral particles, all of which are infectious pathogens.

Moreover, the abuse of large quantities of microbial agents in food production and their wide natural release in the environment through residual waters from humans and animals, as well as the water used in agriculture crops, have severe consequences in the public health which result evident in the case of zoonotic bacteria which cause human diseases transmitted by food.

It is specially preoccupant, although its qualitative and quantitative importance is still uncertain, the possible passing of resistant gens from bacteria from animals to the human pathogens.

In combination with the above described, the excessive use of pesticides in crops has also contributed to the unfortunate acceleration of microbial resistance. Just as antibiotics are insufficient today to eliminate the microbial charge of infectious diseases, antiseptics and disinfectants are also poorly effective to reduce the microbial charges from biological or inert surfaces. Additionally, pesticides are insufficient to battle microbial charges that afflict, destroy complete crops and are causing that various species from the vegetal realm be currently in extinction.

The use of toxic pesticides in crops is added to the dragging of antibiotics contained in animal waste that reach conjunctly the groundwaters, from where the human being is supplied, making this a global alert problem.

Due to the above, it is of vital economic importance for the agriculture and cattle sectors, as well as for the human health, the need of using a new antimicrobial agent capable of being of broad spectrum.

The molecular nanotechnology is an advance so important that its global impact could be comparable to that of the Industrial Revolution, but with the remarkable difference, that in the case of the nanotechnology the huge impact will be evident in a matter of a few years.

In the state of the art are some documents related to the subject of the present invention, such is the case of the International Patent Application No. PCT/KR02/02142 (that corresponds to the International Publication No. WO 2003/059070) which describes a liquid composition to promote the growth of plants, which includes a nanomaterial from titanium dioxide. Said composition contains, as main component, an aqueous solution containing colloids from titanium dioxide. The nanoparticulate titanium dioxide has a particle size such that it can be easily absorbed by plants. The pH from the aqueous solution is adjusted to prevent a fast precipitation of the TiO2 in said aqueous solution, prior dilution of the aqueous solution with water so that the TiO2 have a desired concentration. Also, the composition contains adjuvants for the growth of the plant and a surfactant for dispersion. The composition allows an increase in performance of the crop increasing the photosynthetic efficiency of plants, besides allowing the increase of the bactericide activity from plants against pathogens. Additionally, the composition allows improving the problem of environment pollution caused by the excessive use of biochemical fertilizers and contributes also to an increase in the crop farmers' income.

As can be seen from above, the composition presents the inconvenience that pure TiO2 (non-functionalized) is an inert material and, despite said composition, it requires to have additional nourishing materials, present in the solution. The solution must be a nourishing solution including different oxides or surfactants with particles of TiO2 in suspension, which presents a problem contaminating the soils when in use, by the diluted oxides of heavy metals which are a health problem since they are absorbed by plants of human consumption. It is defined that TiO2 has bactericide activity due to the semiconductor property obtained when exposed to light. It has been proved that TiO2 is not a crop enhancer by itself, as it is an inert material.

Moreover, the Patent MX 339086 describes a functionalized nanoparticulate material of TiO2 prepared with herbal and/or fruit extracts capable of deactivate viruses and eliminate bacteria, fungi, spores, mycobacteria and trypanosomes. The modification (functionalization) at nanometric level in the material of said PCT application, gives to it properties that make it different from the same type materials without said modifications, as well as from the chemical compounds used currently for the applications detected so far.

### SUMMARY OF INVENTION

For the purposes of a better and clearer understanding of the present invention, throughout the description reference is made indifferently to the nanoparticulate compound of titanium dioxide as “nanoparticulate compound of titanium dioxide functionalized”, or, “nanoparticulate compound of titanium dioxide modified with organic functional groups, inorganic radicals and fruit and/or herbal extracts adsorbed on its surface and pores”, but in both cases reference is being made to the same compound. Further still, said nanoparticulate compound is already described and protected in the patent MX 339086.

The present invention refers to various uses of the functionalized titanium dioxide (TiO2) compound mixed with water and, optionally, with at least another material to produce liquid aqueous compositions with a wide microbial applications having different uses and purposes, such that they can be used in the pharmaceutical, food, agriculture, cattle and aquaculture industries, as well as in the medicine.

The nanoparticulate compound of titanium dioxide functionalized is used as water disinfectant and purifier, wherein the percentage of said nanoparticulate compound recommended to achieve the elimination of the different proved microorganisms differ depending on the bacterial population. It is effective in the disinfection of water in samples of different sources with different microorganisms, for example, aerobes mesophilic and fecal and total coliforms, Pseudomonas spp, Acinetobacter spp, Aeromonas and Escherichia co/i, among others, such as is shown in the graph in FIG. 1 of the accompanying drawings, wherein said nanoparticulate compound does not affect the control physical-chemical parameters of water, whereby the treatment process does not require a further step for the elimination thereof. In an additional embodiment of the present invention, the nanoparticulate compound of titanium dioxide functionalized is used as biopesticide in different types of vegetables and seeds, as sterilizer of seeds and as post-harvest disinfectant of various crops to eliminate or reduce the presence of microorganisms and viral particles including largely the bacteria, fungi and viruses groups.

As biopesticide it is used to eliminate the source pollution or sterilization/disinfection of seeds, for example, the case of tomato seeds where it has been observed the latency of Clavibacter michiganensis, or, corn seeds, where the functionalized nanoparticulate TiO2 compound in direct application, or, dosed in emulsions or solutions, proved to be efficient in the reduction of incidences of sick plants.

Additionally, it is used as biopesticide in plantations or crops, wherein through foliar and systemic application in demonstrative plots reducing the incidence of fungi, bacteria and/or viruses in crops, for example and without exclusion, of fruits, green vegetables, perennial pastures, forests, leguminous plants and fruticulture such as papaya, cacao, apple, mango, onion, vanilla, avocado, citrus fruits, chili pepper, corn, coffee, sorghum, alfalfa, pumpkin, potato, exotic woods, walnut trees, cedars, bean, chickpea, roses, orchids, tulips and carnation crops, among others.

It can also be used for disinfection after harvest, it is to say, post-harvest, eliminating the microbial charge and helping to increase the shelf life about 40%, for example and without excluding, papaya, lemon, squash, and tubers. In the case of post-harvest use, the washing or submersion of fruit in the nanoparticulate compound of titanium dioxide functionalized contributes to the increase the products shelf life about 40%, as can be seen in papaya, lemon, pumpkin, and tubers.

In another additional embodiment of the present invention, the nanoparticulate compound of titanium dioxide functionalized is used as preservative to avoid the growth of microorganisms, mainly bacteria and fungi which, even when it has already been mentioned that its activity is not limited thereto. Thanks to the residuality, the microbial effect has an extended action period that allows it to protect food and/or cosmetics from the attack of microorganisms. The nanoparticulate compound of titanium dioxide functionalized can be applied in the food industry as preservative in potted food, canned food, dairy, meat, cheese, fish, prepared food, processed and industrialized food, frozen food and in refrigeration, desserts and beverages, among others, but without being limited thereto.

The nanoparticulate compound of titanium dioxide functionalized can also be used in other industries to protect substances from microbial activity, such is the case of the production of cosmetics or personal care products, for example, in the formulation of lipsticks, color powders and liquids, shampoos, creams, conditioners, soap, toothpastes, but without being restricted to these products.

The nanoparticulate compound of TiO2 is used in tissue regeneration, in different cellular lines and tissues to verify the compatibility, making cytotoxicity and cellular viability studies. Said nanoparticulated titanium dioxide compound is not only selective to pathogenic microorganisms, it also promotes an increase in the proliferative response of tissues with which it is in contact. This induction applied to the treatment of tissues confers the nanoparticulate compound of titanium dioxide functionalized a cicatrizing and/or regenerating cellular effect that has been evaluated in various tissues and cellular lines, even though it is not necessarily restricted thereto.

The nanoparticulate compound of TiO2 can be used in the pharmaceutical industry by administering it through the different systemic pathways, being effective in the treatment and prevention of infectious processes caused by viruses, bacteria, fungi, spores, mycobacteria and parasites. Due to the plurality of microorganisms against which it acts, the nanoparticulate compound of titanium dioxide functionalized could be classified pharmacologically as an anti-infectious o antimicrobial agent.

The nanoparticulate compound of TiO2 is used as microbial agent, being effective in cellular lines infected with different microorganisms.

The nanoparticulate compound of TiO2 is used as antineoplastic, designed to battle pathogenic microorganisms, whose central effect is given in the genetic material by disarticulating the genetic chain, of DNA or RNA. The aforementioned is the same mechanism as that employed in the antineoplastic-cytotoxic drugs, with the advantage that this nanoparticulate material can be targeted specifically to the cancerous cells and can be applied directly on cancerous tumors. The nanoparticulate compound of titanium dioxide functionalized acts at a molecular level by degrading the DNA and RNA molecules of cancer cells following an action mechanism similar to that of elimination of microorganisms, and accordingly, not only eliminates the malignant cells, but it avoids the proliferation of malignant cells. This action is due to the bioselective capacity of the nanoparticulate compound of titanium dioxide functionalized of identifying the negative charges and being attracted thereto. This latter under the universal principle of opposite charges attract each other.

The nanoparticulated compound of TiO2 is used as microbial agent in the livestock, cattle and aquaculture industries, mixing it with various types of commercial foods of the mentioned industries, it was proved that, by being ingested by different animal species, such as shrimp, poultry goats and number of cattle, the mortality ratio decreases mainly in the early stages up to about 33%.

### OBJECTS OF THE INVENTION

Taking into account the disadvantages found in the state of art, it is an object of the present invention to provide various uses of the nanoparticulate compound of titanium dioxide functionalized, as it presents a broad spectrum against viruses, bacteria, fungi, spores, mycobacteria, parasites and against cells with negative charges like those of cancer.

It is another object of the present invention to provide a use of the nanoparticulate compound of titanium dioxide functionalized as water disinfectant, using it either pure or in compositions that allows the elimination in a more efficient way of different microorganisms, particularly pathogens, that could be in water, making it apt for human consumption, in physical-chemical as well as in microbiological parameters.

A further object of the present invention is to provide a use of the nanoparticulate compound of titanium dioxide functionalized as biopesticide, using it either pure or in compositions that allows to eliminate or reduce the presence of microorganisms and viral particles mostly including the groups of bacteria, fungi and viruses in different vegetables and seeds, as seed sterilizer and as post-harvest disinfectant of various crops.

Still a further object of the present invention is to provide a use of the nanoparticulate compound of titanium dioxide functionalized as preservative in the preparation of industrialized hygienic, cosmetic and food products, using it either pure or in compositions for humans as well as cattle and aquaculture industries, wherein the antimicrobial effect is not limited to a time of exposure defined by the disinfection process.

A yet further object of the present invention is to provide a use of the nanoparticulate compound of titanium dioxide functionalized as inductor or activator of tissue regeneration, as it promotes an increase in the proliferative response of tissue with which it is in contact, by using it pure.

Even further, another object of the present invention is to provide a use of the nanoparticulate compound of titanium dioxide functionalized supplemented with herbal and/or fruit extracts, using it either pure or in pharmaceutical compositions due to its effectivity, bioselectivity and innocuousness, being able to be administered through different systemic pathways, being effective in the treatment and prevention of infectious processes caused by viruses, bacteria, fungi, spores, mycobacteria and parasites.

These and other objects, particularities and advantages of the various uses of the nanoparticulate compound of titanium dioxide functionalized of the present patent application, will be apparent for one skilled in the art from the detailed description of certain embodiments and the accompanying figures, as well as the appended claims.

### BRIEF DESCRIPTION OF THE DRAWINGS

The novelty aspects considered characteristics of the present invention will be set particularly in the appended claims. However, the invention itself, as per its organization, as well as its operative method, conjointly with other objects and advantages thereof, will be better understood in the following detailed description of the embodiments of the present invention when read in relation to the accompanying drawings, in which:

FIG. 1 is a graph showing the reduction percentages of fecal (A) and total (B) coliforms, in water.

FIG. 2 is a graph showing the results of tests in control (A) and test (B) demonstrative plots, where is observed a reduction in the affected areas in the different crops, showing additionally the ratio of healthy versus sick plants.

FIG. 3 is a graph showing the increase of mold and yeast tally related to the shelf life of different fruits and vegetables.

FIG. 4 shows results from the thermal analysis in which can be observed that the decomposition occurs in a single stage at a temperature not greater than 200° C. The thermogravimetric and thermo differential analysis show weight loss and decomposition in one step by thermal degradation of the nanoparticulate material.

FIG. 5 shows examples of results in efficacy of tests of preservative effect in food (A) and in cosmetics (B)

FIG. 6 is a graph showing the viability of cellular lines and tissues after contacting with the nanoparticulate compound of titanium dioxide functionalized compared to other active ingredients.

FIG. 7 is a graph showing the lethal dose 50, maximal toxic dose and minimal effective dose.

FIG. 8 is a graph showing the microbial activity of the compositions based on the nanoparticulate compound of titanium dioxide functionalized of the present invention.

FIG. 9 is a graph showing the accumulated mortality during the 48 hours PI (Post-Infection).

### DETAILED DESCRIPTION OF THE EMBODIMENTS OF THE INVENTION

For the purposes of a better and clearer understanding of the present invention, throughout the detailed description reference is made indifferently to the nanoparticulate compound of titanium dioxide functionalized (TiO2), or, to the nanoparticulate titanium dioxide compound modified with functional organic groups, inorganic radicals and fruit and/or herbal extracts adsorbed on its surface and pores, but in both cases reference will be made to the same compound. Further still, said nanoparticulate compound is already described and protected in the patent MX 339086.

In the same way, except in the claims and/or operative examples, unless is expressly specified otherwise, all numeric quantities in the present detailed description referring to material quantities and/or reaction conditions, should be understood as modified by que term “about” in the interpretation of the broader scope of the present invention. According to the above, the present invention refers to, but it not limited to, the quantities of material or reaction conditions expressed within the described numeric limits. Additionally, the description of a group of elements or class of material selected as preferred or adequate to fulfill the objects of the invention, implies that the mixtures of two o more of said group or class elements are equally prepared or adequate.

Now, surprisingly the nanoparticulate compound of titanium dioxide functionalized (TiO2) has been found to, besides the disinfectant and aseptic properties disclosed in the patent MX 339086, have functions and properties as antimicrobial substance due to the following characteristics:
Broad spectrum against viruses, bacteria, fungi, spores, mycobacteria, parasites and against cells with negative charges as those of cancer.
Biodegradability: as it is a nanoparticle with organic vegetable extracts and an inert material of low content it has the specific property of being biodegradable. The above has been proved by laboratory evaluations using established models.
Bioselectivity: as it is a material so small, the effect at cellular level has also been evaluated, finding that neither the sized of the particle nor the superficial modifications damage the cells of the biological medium in which it is used and only acts or attacks the microorganisms. This property is defined as bioselectivity wherein the selectivity is directly related to the superficial functionalization of the material that allows it to chemically identify the pathogenic microorganisms.
Biocompatibility: it is the capacity to perform the desired function according to the medical treatment without interfering or damaging the biological medium in which it is being used, it is to say, it causes no secondary effects in either human, animal or plant users. This makes of it a totally innocuous material, such as the functionalized titanium dioxide, which has no secondary, adverse or toxicological, nor cytotoxicoloical effects. The above, provides the advantage of not being toxic. This has been proved with evaluations of acute, subchronic and chronic expositions through different systemic administration pathways without presenting effects on the normal functions of the test organisms. These tests have been made by means of the models approved by the OECD (Organization for Economic Co-operation and Development).
The use of this compound does not cause resistance in the microorganisms, due to the degradation mechanism of the genetic material, either DNA or RNA, occurs by que dissolution of peptide bonds, C—C bonds and C—N bonds in nucleic acids. The chemical attraction that promotes the molecular approaching is due to the functional groups adsorbed on the surface, and the rupture due to the Lewis and Bronsted acid sites of the surface of metallic oxide. With this action mechanism the capacity of the microorganism, viral particle or cell with negative charge to pass on to the next generation the genetic information on how to resist said substance is nullified.

Thus, its wide microbial action is due to a double action mechanism, acting on the cellular membrane of microorganisms that have one, for example, bacteria and fungi, but at the same time acting on the genetic material. This second point is the one serving as mechanism in deactivating viruses that, unlike other microorganisms, don't have membrane.

Likewise, as it is known from the referred patent MX 339086, the nanoparticulate functionalized titanium dioxide material has the following general formula:

E/MaO2(c)(OH)v(PO4)w(SO4)xCly(NH2)z (1)

where E is a complex solution of fruits and/or herbal extracts and M is titanium. The fruit and/or herbal extracts are selected from the group comprising grapefruit, lemon, tangerine, among other citrus fruits.

According to the above, in the present invention various uses of the nanoparticulate compound of titanium dioxide functionalized are described, which is preferred, but is not limited to be used pure (100%) or in solutions containing said compound as active ingredient in an effective quantity from 10% to 90% for products requiring dilution.

In an additional embodiment of the present invention, the nanoparticulate compound of titanium dioxide functionalized can be used in low proportions where said compound is found in an effective quantity of up to 1%. The concentration used will depend on the dilution specified in the final product, such that a final concentration from 0.125% up to 0.625% is guaranteed.

The compound of functionalized titanium dioxide (TiO2) is mixed with water and, optionally, with at least another material to produce liquid aqueous compositions with a wide microbial application having different uses and purposes, such that they can be used in the pharmaceutical, food, agriculture, cattle and aquaculture, as well as medical industries.

A. Use of the Nanoparticulate Compound of Titanium Dioxide Functionalized as Water Disinfectant or Purifier:

Potabilization refers to water being apt for human consumption in its physical-chemical parameters and in the microbiological specification, water disinfection refers merely to the deactivation of microorganisms, specially of pathogens, causing diseases in the water consumers, wherein the intensity and severity will vary depending on many factors as microorganism type and concentration in water of the infectious agent. The microbiological disinfection is perhaps the most important treatment and of major transcendence in water potabilization.

In a particularly preferred embodiment of the present invention, the nanoparticulate compound of titanium dioxide functionalized is used as water disinfectant or purifier, using preferably a water-base suspension whose percentage of said nanoparticulate compound of titanium dioxide functionalized as active ingredient is present in an effective quantity from 30% to 99%. The percentage recommended to achieve the elimination of the different tested microorganisms vary depending on the bacterial population.

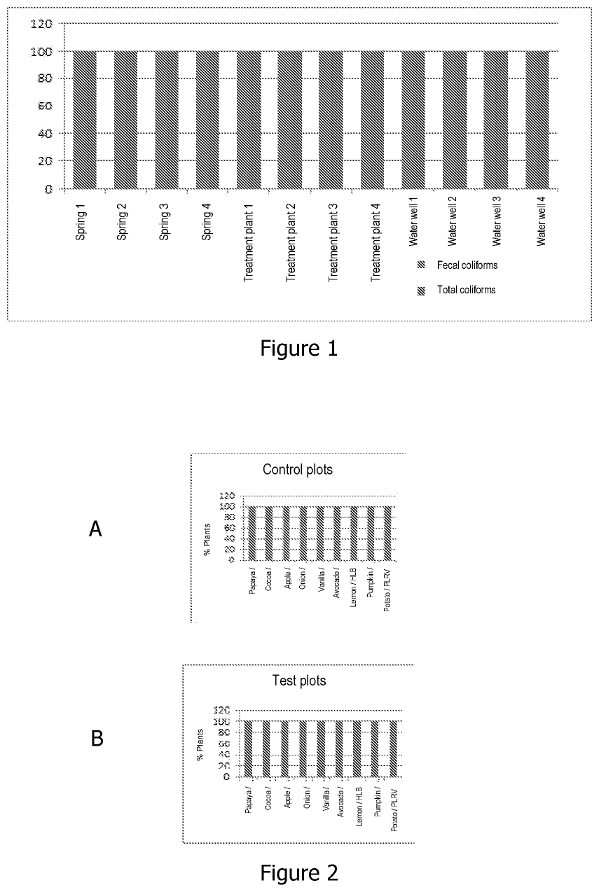

The tests made on the use of the nanoparticulate compound of titanium dioxide functionalized show a reduction of about 99.999% of the microorganisms present in water, from residual water treatment plants, as well as from springs and wells pertaining to the potable water distribution network. Its efficacy in water disinfection in samples of the different mentioned sources with different microorganisms, for example, aerobes mesophilic and fecal and total coliforms, Pseudomonas spp, Acinetobacter spp, Aeromonas and Escherichia coli, among other has been proved, as is shown in the graph in FIG. 1 of the accompanying drawings.

The tests show that the use of the nanoparticulate compound of titanium dioxide functionalized does not affect the physical-chemical parameters of water control, whereby the treatment process does not require a posterior step for the elimination thereof.

Moreover, by acting on the genetic material of the microorganisms a “natural” death is produced, avoiding releasing of endotoxins in water. Tests of biodegradability performed to the nanoparticulate compound of titanium dioxide functionalized show that, when being in contact with biotic and abiotic factors of the environment, said nanoparticulated compound is biodegradable in a 100%.

In table 1 water and measured contaminant parameters are shown, as turbidity, pH, true color, temperature, conductivity, total solids, total dissolved solids, residual chlorine, phenolphthalein alkalinity, total alkalinity, bicarbonates, carbonates, hydroxides, total hardness, sulphates, chlorines, total coliforms and fecal coliforms. It is important to note that the physical-chemical parameters of water, as well as the conductivity, were not altered and they are reflected in said table 1.

TABLE 1
Physical-chemical and microbiological tests from different sources of water, before
and after the application of the formulations of the present invention.

| | | WATER TREATMENT | | | | | |
|---|---|---|---|---|---|---|---|
| | | PLANT | | DAM | | SPRING | |
| | | | POST- | | POST- | | POST- |
| PARMETER | UNIT | PRIOR | APPLICATION | PRIOR | APPLICATION | PRIOR | APPLICATION |
| Turbidity | UTN | 9.7 | 7.5 | 6 | 5.5 | 4.5 | 3 |
| pH | U | 7.11 | 7.25 | 7.32 | 7.39 | 7.42 | 6.92 |
| Ture color | U Pt/Co | 41 | 23 | 25 | 19 | 16 | 9 |
| Temperature | ° C. | 20.2 | 20.2 | 20.1 | 20.5 | 20.6 | 20.6 |
| Conductivity | μmhos/cm | 177.9 | 173.6 | 157.2 | 152.6 | 151.3 | 151.9 |
| Total solids | mg/l | 312 | 238 | 214 | 180 | 172 | 86 |
| Total disolved | mg/l | 73.2 | 69.2 | 82.6 | 78.7 | 75.7 | 67.2 |
| solids | | | | | | | |
| Residual | mg/l | 0 | 0 | 0 | 0 | 0 | 0 |
| chlorine | | | | | | | |
| Phenolphthalein | mg/l | 0 | 0 | 0 | 0 | 0 | 0 |
| alkalinity | | | | | | | |
| Total alkalinity | mg/l | 164 | 124 | 126 | 92 | 98 | 92 |
| Bicarbonates | mg/l | 164 | 124 | 126 | 92 | 98 | 89 |
| Carbonates | mg/l | 0 | 0 | 0 | 0 | 0 | 0 |
| Hydroxides | mg/l | 0 | 0 | 0 | 0 | 0 | 0 |
| Total hardness | mg/l | 126 | 136 | 240 | 242 | 70.2 | 76.9 |
| Sulphates | mg/l | 6.7 | 7.47 | 7.09 | 7.09 | 8.19 | 6.15 |
| Chlorines | mg/l | 11.5 | 3.5 | 3.5 | 4.5 | 6 | 6.7 |
| Total coliforms | NMP/100 ml | 4.6 | 0 | 3.1 | 0 | 4.6 | 0 |
| Fecal coliforms | NMP/100 ml | 6.1 | 0 | 4.7 | 0 | 1.1 | 0 |

Comparing the use of the nanoparticulated functionalized titanium dioxide compound in water potabilization with the traditional method of chlorination, it is clearly visible that there is a microbiological elimination of 100% in fecal coliforms and of 100% in aerobes mesophilic, against 87% to 99% of said chlorination method, but without toxicity and collateral damages from the use and abuse of chlorinated substances.

It should be noted that the chlorination method is bond to hepatic damage processes, asthma and heavy metal accumulation, while toxicity tests on nanoparticulated functionalized titanium dioxide compound prove its innocuousness. As shown by the results in table 1, the physical-chemical variables do not vary between prior and post-application sample collections of said nanoparticulate compound of functionalized TiO2, demonstrating the microbiological effectivity and no alteration of physical-chemical characteristics.

In table 2 colony forming units from different microorganisms present in water from various sources are shown. In the pre- and post-application data from nanoparticulated compound of functionalized TiO2 the efficacy of said nanoparticulated compound is quantified by eliminating the totality of pathogens, which is equally demonstrated in FIG. 1.

TABLE 2
Shows the 100% effectivity of the nanomaterial
against various microorganisms

| Microorganisms | Prior UFC/ml | Post UFC/ml |
|---|---|---|
| Total coliforms totales | 400 | 0 |
| Fecal coliforms fecales | 100 | <1.1 |
| Pseudomonas spp, | 800 | 0 |
| Acinetobacter spp, | 996 | 0 |
| Aeromonas | 432 | 0 |
| Escherichia coli | 1,267 | 0 |

These unique characteristics, confer the nanoparticulated titanium dioxide compound modified with organic functional groups, inorganic radicals and fruit and/or herbal extracts adsorbed on its surface and pores, the viability of being used as active ingredient in liquid, colloidal and solid disinfectant products for water treatment, in the process of disinfection thereof, through the elimination of microorganisms, properties which were believed to be impossible, since it was believed that the material would loss its properties in large amounts of water.

B. Use of the Nanoparticulate Compound of Titanium Dioxide Functionalized as Biopesticide and Post-Harvest:

Due to their ways of application, pesticides can be classified as of superficial use, since it merely acts on the external surface, or, systematically when there is absorption and it is transported towards the plant's interior.

In an additional aspect of the present invention, the nanoparticulate compound of titanium dioxide functionalized is used as biopesticide and post-harvest, where, said nanoparticulated compound is used mixed with water in an effective quantity from 0.8% to 30%, preferable from 0.8% to 15%; additionally, the use comprises adding a ionic surfactant agent in an amount of up to 4% and an organic or inorganic foliar adherent in an amount of up to 6%, which allows the nanoparticulate compound of titanium dioxide functionalized to remain adhered to the plants leaves and to extent its effect, wherein the organic foliar adherent is preferred to be, but without being limited to resins or polymers, while the inorganic foliar adherent is preferred to be, but without being limited to acrylates.

The nanoparticulate compound of titanium dioxide functionalized application as pesticide was evaluated with different types of vegetables and seeds, as seed sterilizer and as post-harvest disinfectant of various crops to eliminate or reduce the presence of microorganisms and viral particles including mainly the groups of bacteria, fungi and viruses. These are those frequently found in the agriculture and food industries in the different process stages causing problems from the crops and/or livestock, to even affecting productive processes, shelf life and human being's life.

In a first alternative embodiment of the use of the nanoparticulate compound of titanium dioxide functionalized as pesticide, it is for eliminating the source of contamination or sterilization/disinfection of seeds, for example, the case of tomato seeds where Clavibacter michiganensis latency was observed, or, corn seeds, where the direct application of the nanoparticulated functionalized titanium dioxide compound, or, dosed in emulsions or solutions, proved to be efficient in reducing incidences of sick plants. The comparative results of studies in demonstrative plots are shown in the graph in FIG. 2 of the accompanying drawings, showing in red the proportion of healthy plants.

In a second alternative embodiment of the use of the nanoparticulate compound of titanium dioxide functionalized as biopesticide, it is on plantations or crops, wherein through the foliar and systemic application on demonstrative plots, reductions of up to 100% of efficacy in incidences of fungi, bacteria and/or viruses have been reported on crops, for example and without excluding, as fruit, green vegetables, perennial pastures, forests, leguminous plants crops and fruticulture such as papaya, cacao, apple, mango, onion, vanilla, avocado, citrus fruits, chili pepper, corn, coffee, sorghum, alfalfa, pumpkin, potato, exotic woods, walnut trees, cedars, bean, chickpea and crops of roses, orchids, tulips and carnations, among others.

The nanoparticulate compound of titanium dioxide functionalized effectivity results as pesticide against various diseases and plagues in different types of crops, for example, Solanaceae, tubers, berries, fruits, floriculture, orchids, among others, are shown in table 3, in which the efficiency in reduction can be seen going from 93% to 100% in incidences of fungi, bacteria and/or viruses in the crops.

TABLE 3
Studies comparative results in demonstrative plots

| Plant/disease or | Control | Affected | Healthy | Test | Affected | Healthy |
|---|---|---|---|---|---|---|
| microorganisms | plots | plants | plants | plots | plants | plants |
| Papaya/Ringspot virus | 100 | 77 | 23 | 100 | 2 | 98 |
| Cocoa/Moniliasis | 100 | 78 | 22 | 100 | 3 | 97 |
| Coffe/Coffe rust | 101 | 62 | 39 | 101 | 3 | 98 |
| Apple/Scab of apple | 100 | 68 | 32 | 100 | 2 | 98 |
| Onion/Pyrenochaeta | 100 | 69 | 31 | 100 | 1 | 99 |
| terrestis | | | | | | |
| Vanilla/Antracnosis | 100 | 76 | 24 | 100 | 1 | 99 |
| Avocado/Phytophthora | 100 | 79 | 21 | 100 | 2 | 98 |
| cinnamomi | | | | | | |
| Lemon/HLB | 100 | 74 | 26 | 100 | 3 | 97 |
| Pumpkin/Antracnosis | 100 | 60 | 40 | 100 | 1 | 99 |
| Potato/PLRV | 100 | 63 | 37 | 100 | 1 | 99 |

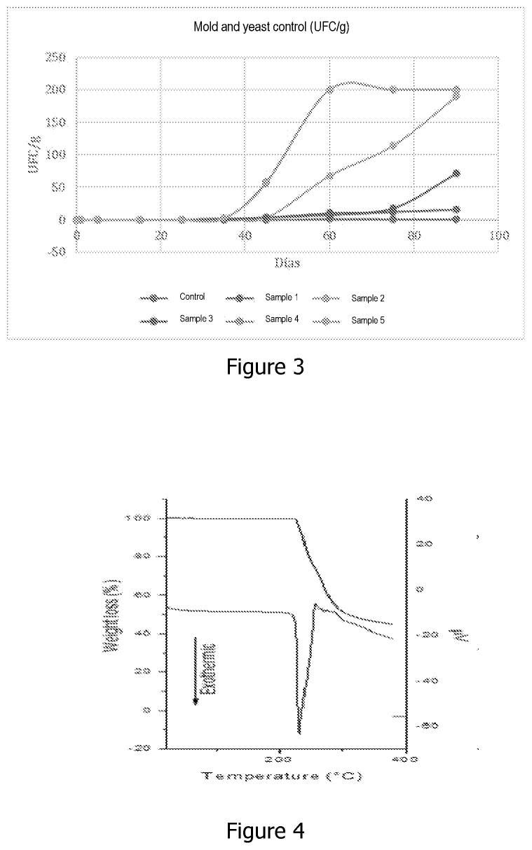

In an additional aspect of the present invention, the nanoparticulate compound of titanium dioxide functionalized is used for disinfecting after the harvest, it is to say, post-harvest, eliminating the microbial charge and helping to increase shelf life about 40%, for example and without excluding, of papaya, lemon, pumpkin, and tubers. In the graph in FIG. 3 of the accompanying drawings the increase of shelf life of different fruits and vegetable is shown. In the case of post-harvest use, washing and submersion of fruit in the nanoparticulated functionalized titanium dioxide compound, shown to contribute to increase the shelf life of products to up to about 40%, as can be observed in papaya, lemon, pumpkin, and tubers, such as shown in table 4.

In another additional aspect of the present invention, the nanoparticulate compound of titanium dioxide functionalized is present in an oily formulation in an effective quantity from 70% to 90% for improving the absorption by plants.

TABLE 4
Shelf life increased in different fruits and vegetables

| | Time in days under normal | | |
|---|---|---|---|
| | environmental conditions | | |
| Fruit or | | Nanomaterial | Increased shelf |
| vegetable | Control | applied | life percentage |
| Papaya | 7 | 13 | 86 |
| Cocoa | 28 | 40 | 43 |
| Coffe | 28 | 54 | 93 |
| Apple | 5 | 7 | 40 |
| Onion | 5 | 7 | 4C |
| Vanilla | 15 | 24 | 60 |
| Avocado | 15 | 18 | 20 |
| Lemon | 15 | 21 | 40 |
| Pumpkin | 4 | 7 | 75 |
| Potato | 14 | 21 | 50 |

C. Use of Nanoparticulate Compound of Titanium Dioxide Functionalized as Preservative:

Derived from the aforementioned use, the application of the nanoparticulate compound of titanium dioxide functionalized as a preservative in the preparation of hygienic products, cosmetics and industrialized food was tested, both for humans and for the livestock and aquaculture industry, where the effect antimicrobial is not limited to an exposure time defined by the disinfection process.

In another additional aspect of the present invention, the nanoparticulate compound of titanium dioxide functionalized is used as preservative, where said nanoparticulate compound is mixed with water in an effective quantity from 0.02% to 5%. The most important properties are the microbial activity, residuality and thermal stability of the nanoparticulate compound of titanium dioxide functionalized.

The use of the nanoparticulate compound of titanium dioxide functionalized as preservative, strictly refers to its effectivity to avoid the microorganisms' growth, mainly bacteria and fungi which, even though already mentioned above, activity is not limited thereto. Due to residuality, the microbial effect has an extended action period which allows it to protect food and/or cosmetics from the attack of microorganisms.

The use of nanoparticulate compound of titanium dioxide functionalized was evaluated by differential thermal and thermogravimetric analysis, with which the nanoparticulate compound of titanium dioxide functionalized was proved to remain stable in a temperature interval going from −10° C. to 200° C., whereby it can be used in processes that go from being preserved in refrigeration or frozen to being cooked without modifying its structure or affecting the food in question. These properties can also be beneficial to other industries, as the chemical, pharmaceutical or cosmetic to be kept in good conditions or to extent the average life of products and raw material.

The nanoparticulate compound of titanium dioxide functionalized can be applied in the food industry as preservative in potted food, canned food, dairy, meat, cheese, fish, prepared food, processed and industrialized food, food in refrigeration and frozen, desserts and beverages, but without being limited thereto.

The nanoparticulate compound of titanium dioxide functionalized can also be used in other industries to protect substances from microbial activity, such is the case of cosmetic or personal care products production, for example, in the formulation of lipsticks, color powder and liquids, shampoos, creams, conditioners, soaps, toothpastes, without being restricted only to these products.

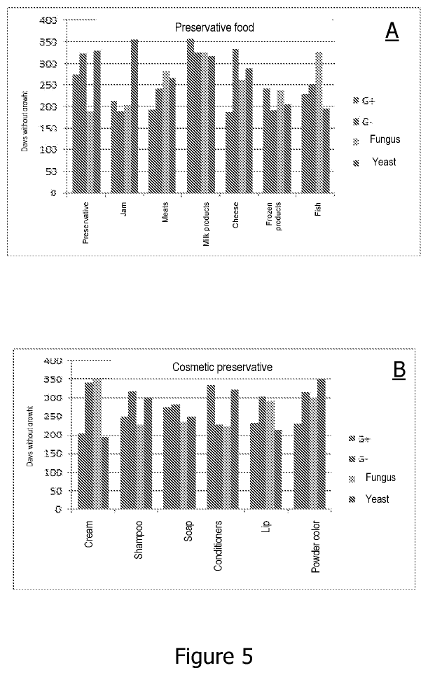

The thermal analysis results are shown in the graph in FIG. 4 in the accompanying drawings, in which decomposition can be observed to occur in a single stage at a temperature higher than 200° C. The examples of efficacy results as preservative are shown in the graph in FIG. 5 in the accompanying drawings.

D. Use of Nanoparticulate Compound of Titanium Dioxide Functionalized as Inductor or Activator of Tissue Regeneration:

The engineering of tissues changed the field of biomaterial development and refers to the practice of combining biologically active scaffolds, cells and molecules to create functional tissues. The purpose of the engineering of tissues is to recollect ideas or theories that restore, maintain or improve damaged tissues or complete organs. Artificial skin and cartilage are examples of tissues fabricated by engineering which have been approved by the FDA. However, currently they have a limited usage in human patients.

Tests were made with the nanoparticulate compound of titanium dioxide functionalized with different cellular lines and tissues to verify the compatibility, performing studies of cytotoxicity and cellular viability. Studies were performed to compare the use of said nanoparticulate compound of titanium dioxide functionalized against disinfectants or antiseptics of common use in the medical practice. Said studies show that the nanoparticulated compound of titanium dioxide is not only selective to pathogenic microorganisms, but it also promotes an increase in proliferative response of the tissues with which it is contacted. This induction applied to the treatment of tissues confers the nanoparticulate compound of titanium dioxide functionalized a cicatrizing and/or cellular regeneration effect which has been evaluated in various tissues and cellular lines, while not necessarily it is restricted thereto.

In this aspect of the invention the nanoparticulate compound of titanium dioxide functionalized can be used in low proportions where said compound is in an effective quantity of up to 1%. The concentration used will depend on the dilution specified on the final product, such that the final concentration from 0.125% to 0.625% is guaranteed.

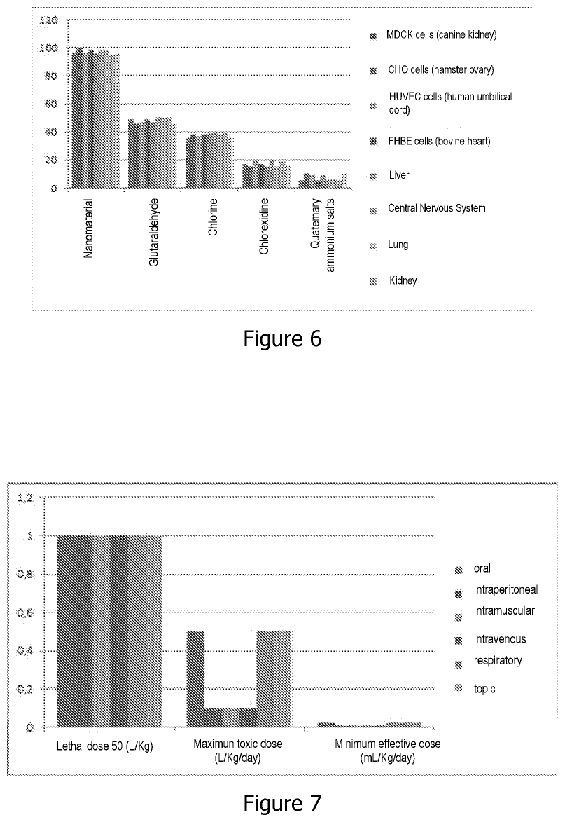

In the graph in FIG. 6 of the accompanying drawings the tested cellular lines, tissues and cellular viability results are shown showing proliferation and activation.

E. Use of Nanoparticulate Compound of Titanium Dioxide Functionalized in the Pharmaceutical Industry:

A drug is known to be a chemical substance used for treatment, cure, prevention o diagnosis of a certain disease or for also inhibiting the beginning of an undesired physiological process. Then the drug's remarkable and characteristic feature will be to be a substance applied to the body in an exogenic way and that will cause an imminent change in the cellular activity, such is the purpose of its, particularly medical, usage.

Derived from the effectiveness, bioselectivity and innocuousness evaluations of the nanoparticulated compound of titanium dioxide functionalized, it is allowed to be administered by different systemic pathways, being effective in the treatment and prevention of infectious processes caused by viruses, bacteria, fungi, spores, mycobacteria and parasites. Due to the plurality of microorganisms against which it acts, the nanoparticulate compound of titanium dioxide functionalized has been pharmacologically classified as an anti-infectious or antimicrobial agent. It should be noted that this classification is of greater importance than that of an antibiotic, since it includes antiviral, antifungal/antimycotic, antimycobacterial, anti-sporulating and antiparasitic action.

The specific action through which a drug, compound, molecule or antimicrobial (among which is the nanoparticulated compound of functionalized titanium dioxide) reaches its efficacy, is known a “action mechanism”, which can be a biochemical process, an enzymatic reaction, a movement of electrical charges, a movement of Ca2+ through the membranes or a catalytic process that causes an observable and quantifiable effect.

Currently existing microbial agents act through a series of mechanisms, very different among them and whose targets are in different regions of the cell under attack. The various regions of microbial attack are generally considered: cellular wall, cellular membrane, protein synthesis and nucleic acid synthesis.

For the use of the nanoparticulate compound of titanium dioxide functionalized in the pharmaceutical industry, tests were made to determine the lethal dose and dose 50 (LD and D50). In the graph in FIG. 7 of the accompanying drawings the lethal dose 50, toxic maximal dose and effective minimal dose are shown evaluated. In this aspect of the invention, the nanoparticulate compound of titanium dioxide functionalized (TiO2) can be used in low proportions where said compound is in an effective quantity of up to 1%, wherein the concentration used will depend on the dilution specified on the final product, such that a final concentration going from 0.125% to 0.625% is guaranteed.

F. Use of Nanoparticulate Compound of Titanium Dioxide Functionalized as Microbial Agent:

Generally, the microbial substances are chemical compounds of fermentative, biosynthetic origin or derived from chemical synthesis, whose effect is the decrease in number of bacteria, mycoplasmas, fungi or protozoan present in animal, vegetal species or the human being, as well as in inert surfaces.

Antimicrobials can be classified according to the microorganism on which they act as:
Antibacterial: that inhibiting or reducing the quantity of bacteria present;
Antifungal/antimycotic: that inhibiting or reducing the quantity of fungi present;
Antiviral: that inhibiting or reducing the quantity of viral particles present:
Antimycobacterial: that inhibiting or reducing the quantity of mycobacteria present; and
Antiparasitic: that inhibiting or reducing the quantity of parasites present.

In another additional aspect of the present invention, the nanoparticulate compound of titanium dioxide functionalized is used as microbial agent, wherein said nanoparticulated compound is mixed with water in an effective quantity from 0.00025% to 34.97%.

For the use of nanoparticulate compound of titanium dioxide functionalized, initially, efficacy tests were made on cellular lines infected with different microorganisms, such as VERO (African monkey kidney) confluent cells, dog's kidney or MDCK (Madin-Darby Canine Kidney), as well as necessary tests were made through the research protocols in small species using the methodologies approved by the OECD guidelines for the evaluation for toxicity, cytotoxicity, irritation, absorption or lethal dose, among others.

Preliminary in vitro tests with human cellular lines demonstrated antifungal, antibacterial, antiviral or virucide, mycobactericide and antiparasitic activity. For example, a case diagnosed with lung candidiasis, which was treated with fluconazole, ingestible nanoparticulate compound of titanium dioxide functionalized was used and in 3 days the fungus was eradicated.

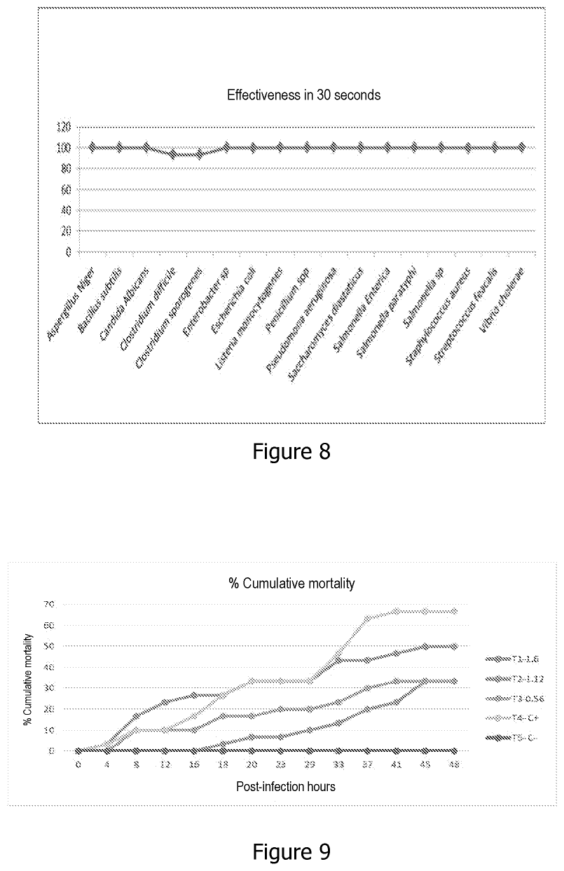

Another case was the treatment of tuberculosis and non-tuberculosis mycobacteria, where nanoparticulate compound of titanium dioxide functionalized pure and with a topic colloidal formulation was used, eradicating Mycobacterium avium and Mycobacterium abcessus pustules. In the graph in FIG. 8 of the accompanying drawings the anti-infectious activity from various microorganisms is shown.

Gastrointestinal cases as Helicobacter pylori or Escherichia coli were initially treated with liquid formulations in suspension form, with concentrations of the nanoparticulate compound of titanium dioxide functionalized from 80 ppm to 300 ppm, where the proved effective doses were from 0.001 ml to 2.1 ml per kilogram of body weight.

G. Use of nanoparticulate compound of titanium dioxide functionalized as antineoplastic.

Antineoplastic is a substance that avoids the development, growth or proliferation of malignant tumor cells. These substances can be of natural, synthetic or semisynthetic origin.

The action mechanism from antineoplastic drugs is characterized by affecting processes in cellular division. The alkylate agents that form bonds with DNA that prevent the replication and transcription of RNA are the most effective. They can act in any phase of the cellular cycle but are cytotoxic and can cause collateral effects on reproductive organs and cancer in other tissues. The antimetabolic drugs can also act on the synthesis process of the cellular cycle, specifically on DNA and RNA synthesis by integrating into their molecules, thereby avoiding the correct transcription and replication thereof. These drugs effects are not specific, since they are no targeting cancerous cells or tumors, which causes collateral effects on the genetic material of healthy cells, resulting in irreversible damage to the organism.

Moreover, nanotechnology is an emerging area having applications on the medical field, being one the designed and synthesis of nanoparticles against cancer. The advantage offered by these innovations, not strictly drugs, are that they can be manipulated to be targeted specifically to a type of cancer o cancerous tumor. Due to the above, the nanoparticulate compound of titanium dioxide functionalized is a biotechnological product designed to battle pathogenic microorganisms, whose central effect is done on the genetic material by disarticulating the genetic chain of DNA or RNA. The above is the same mechanism as the one employed on the antineoplastic-cytotoxic drugs, having as advantage that this nanoparticulate material can be targeted specifically to the cancerous cells and can be directly applied to cancerous tumors.

Other tests were made by the TUNEL staining methodology on tissues extracted from cerebral tumors induced on Wistar rats, by means of an animal model of multiform glioblastoma which generate cancerous tumors on rats comparable to those produced on humans by the disease. In these tests the nanoparticulate compound of titanium dioxide functionalized is observed to act at molecular level by degrading DNA and RNA molecules from cancerous cells following an action mechanism similar to that of microorganisms' elimination, and consequently not only eliminates malignant cells, but avoids the proliferation of malignant cells. This action is due to the nanoparticulate compound of titanium dioxide functionalized bioselective capacity of identifying the negative charges and of being attracted by them. This latter being under the universal principle of opposite charges attract each other.

In this aspect of the invention the nanoparticulate compound of titanium dioxide functionalized (TiO2) can be used in low proportions where said compound is in an effective quantity of up to 1%. The concentration used will depend on the dilution specified on the final product, such that a final concentration from 0.125% to 0.625% is guaranteed.

H. Use of Nanoparticulate Compound of Titanium Dioxide Functionalized as Antimicrobial Agent for the Livestock, Cattle and Aquaculture Industries:

The constant resistance of microorganisms to antibiotics has led the researchers to develop new molecules for controlling infections caused by pathogens. From various studies made where nanoparticulate compound of titanium dioxide functionalized was mixed with various types of commercial food items from said industries, it was proved that, when ingested by different animal species, such as shrimp, poultry, goats and cattle heads, mortality rate decreases mainly on early stages to about 33%. For example, comparative bioassays made with white shrimp larvae with a determined quantity of added nanoparticulate material versus other food, demonstrated to have a preventive effect against Vibro paraheaolyticus and the larvae mortality rate is decreased 33% adding 14 ml/kg of food.

Histopathologic, polymerase chain reaction tests, physical-chemical, bacteriologic and toxicologic analysis proved that there was no alteration, secondary or adverse reactions, also there was no toxicologic effect on the digestive, respiratory or nervous systems.

In the graph in FIG. 9 of accompanying drawings, the accumulated mortality is shown where T1-1.6 is food with a minimal dose of the nanoparticulate compound of titanium dioxide functionalized to 100%, T2-1.12 is food with the ml of effective dose of the nanoparticulate compound of titanium dioxide functionalized to 100%, T3-0.56 is food with excessive dose of the nanoparticulate compound of titanium dioxide functionalized to 100%, T4-C+ is the positive control, and T5-C− the negative control.

While reference has been made in the above description to various uses of the nanoparticulate compound of titanium dioxide modified with organic functional groups, inorganic radicals and fruit and/or herbal extracts adsorbed on its surface and pores of the present invention, it should be emphasized that are possible numerous modifications to said uses, but without departing from the true scope of the invention, such that the technical characteristics of the present invention described in said uses of the invention, which are claimed by the claims, can be used individually or in any arbitrary combination for the making of said present invention, as well as different uses that had not been described herein. Accordingly, it should be understood that the uses of the present invention are merely illustrative and are not intended to limit the scope of the present invention, unless by that established by the state of the art and the appended claims.

1-24. (canceled)

25. A method of protecting a plant or its seeds from pests, contamination, and/or microbes, the method comprising:
applying a nanoparticulate compound to a plant or its seeds,
wherein the nanoparticulate compound is titanium dioxide modified with functional organic groups, inorganic radicals, and fruit and/or herbal extracts adsorbed on its surface and in its pores.

26. The method of claim 25, wherein the plant is a crop,
wherein the applying is a systemic or foliar application, and
wherein the incidence of fungi, bacteria, and/or viruses on the plant is reduced.

27. The method of claim 26, wherein the plant is at least one selected from the group consisting of a fruit, a green vegetable, a perennial pasture plant, a forest plant, and a leguminous plant.

28. The method of claim 25, wherein the nanoparticulate compound is applied directly to the plant or its seeds without dilution.

29. The method of claim 25, wherein the nanoparticulate compound is a component of an emulsion or a solution,
wherein the emulsion or solution comprises:
the nanoparticulate compound at a concentration of 0.8-30 wt %;
an ionic surface-active agent at a concentration of up to 4 wt %; and
an organic or inorganic foliar adherent at a concentration of up to 6 wt %, each relative to a total weight of the emulsion or solution.

30. The method of claim 29, wherein the nanoparticulate compound is present in the emulsion or solution at a concentration of 0.8-15 wt %.

31. The method of claim 25, wherein the nanoparticulate compound is present in an oily formulation at a concentration of 70-90 wt % relative to the total weight of the oily formulation.

32. The method of claim 29, wherein an organic foliar adherent is present, and
wherein the organic foliar adherent is a resin or a polymer.

33. The method of claim 29, wherein the organic or inorganic foliar adherent comprises an acrylate.

34. A method of inhibiting microbes, the method comprising:
applying a nanoparticulate solution to at least one selected from the group consisting of a hygienic product, a cosmetic product, and a food product,
wherein the nanoparticulate solution comprises water and 0.02-5 wt % of a nanoparticulate compound,
wherein the nanoparticulate compound is titanium dioxide modified with functional organic groups, inorganic radicals, and fruit and/or herbal extracts adsorbed on its surface and in its pores, and
wherein the inhibition of microbes as a result of the applying is not limited by the exposure time of the applying.

35. A method of inducing or activating tissue regeneration, the method comprising:
contacting a tissue with a nanoparticulate compound,
wherein the contacting increases a proliferative response of the tissue or creates a cicatrizing and/or cellular regeneration effect in the tissue, and
wherein the nanoparticulate compound is titanium dioxide modified with functional organic groups, inorganic radicals, and fruit and/or herbal extracts adsorbed on its surface and in its pores.

36. The method of claim 35, wherein the nanoparticulate compound is a component of a composition, and
wherein during the contacting the nanoparticulate compound is present in the composition at a concentration of 0.125-0.625 wt % relative to the composition.

37. A method of treating an infection in a human or an animal with a nanoparticulate compound, the method comprising:
administering the nanoparticulate compound through a systemic pathway of the human or the animal,
wherein the nanoparticulate compound is titanium dioxide modified with functional organic groups, inorganic radicals, and fruit and/or herbal extracts adsorbed on its surface and in its pores, and
wherein the infection is caused by at least one selected from the group consisting of a virus, a bacterium, a fungus, a spore, a mycobacterium, and a parasite.

38. The method of claim 37, wherein the nanoparticulate compound is a component of a composition, and
wherein during the administering the nanoparticulate compound is present in the composition at a concentration of 0.125-0.625 wt % relative to the composition.

39. A method of treating a pathogenic microorganism, the method comprising:
contacting a pathogenic microorganism with a nanoparticulate compound,
wherein the nanoparticulate compound disarticulates the DNA or RNA of the pathogenic microorganism, and
wherein the nanoparticulate compound is titanium dioxide modified with functional organic groups, inorganic radicals, and fruit and/or herbal extracts adsorbed on its surface and in its pores.

40. The method of claim 39, wherein the nanoparticulate compound is a component of a composition, and
wherein during the contacting the nanoparticulate compound is present in the composition at a concentration of 0.125-0.625 wt % relative to the composition.

41. A method of reducing a mortality rate in aquaculture or livestock, the method comprising:
mixing a nanoparticulate compound in a food consumed by the aquaculture or the livestock,
wherein the nanoparticulate compound is titanium dioxide modified with functional organic groups, inorganic radicals, and fruit and/or herbal extracts adsorbed on its surface and in its pores, and
wherein the mortality rate of the aquaculture or the livestock is decreased by up to 33% after the food is consumed.
